(19) United States
(12) Patent Application Publication
(10) Pub. No.: US 2025/0302601 A1
(43) Pub. Date: Oct. 2, 2025

(54) METHOD AND APPARATUS FOR FIXATION OF IMPLANTABLE DEVICES ADJACENT A BODY LUMEN

(71) Applicant: Uromedica, Inc., Plyymouth, MN (US)

(72) Inventors: Timothy C. Cook, Wayzata, MN (US); John H. Burton, Minnetonka, MN (US)

(21) Appl. No.: 19/176,983

(22) Filed: Apr. 11, 2025

Related U.S. Application Data
(63) Continuation of application No. 16/532,811, filed on Aug. 6, 2019, now Pat. No. 12,295,824.
Application No. 16/532,811 is a continuation of application No. 15/371,591, filed on Dec. 7, 2016, now abandoned.
Application No. 15/371,591 is a continuation of application No. 15/143,784, filed on May 2, 2016, now abandoned.
Application No. 15/143,784 is a continuation of application No. 14/169,988, filed on Jan. 31, 2014, now abandoned.
Application No. 14/169,988 is a continuation of application No. 12/467,257, filed on May 16, 2009, now abandoned.
(60) Provisional application No. 61/053,758, filed on May 16, 2008.

(51) Int. Cl. A61F 2/00; A61M 25/00

(52) U.S. Cl. CPC A61F 2/0027; A61F 2/004; A61F 2/0077; A61M 25/0017; A61F 2002/0086; A61F 2250/0018

(57) ABSTRACT

The present application relates to method and apparatus for reducing migration and rotation of implantable devices including an expandable element and an elongate portion.

### CLAIM OF PRIORITY

The present application is a continuation of U.S. patent application Ser. No. 16/532,811, filed on Aug. 6, 2019, which is a continuation of U.S. patent application Ser. No. 15/371,591, filed on Dec. 7, 2016, which is a continuation of U.S. patent application Ser. No. 15/143,784, filed on May 2, 2016, which is a continuation of U.S. patent application Ser. No. 14/169,988, filed on Jan. 31, 2014, which is a continuation of U.S. patent application Ser. No. 12/467,257, filed on May 16, 2009, which claims the benefit under 35 U.S.C. 119 (e) of U.S. Provisional Patent Application Ser. No. 61/053,758, filed on May 16, 2008, which applications are incorporated herein by reference in their entireties.

### FIELD OF THE INVENTION

The present subject matter relates to method and apparatus for fixation of implantable devices adjacent a body lumen.

### BACKGROUND

The implantation of medical devices into the tissue of patients is complicated. The surgery can be partially or wholly ineffective if the implanted devices migrate or rotate within the body after implantation. Such devices need to be removed and reimplanted if possible. Each time a device is surgically implanted or explanted, the patient suffers risks of trauma and infection, which can sometimes be lethal to the patient.

There is a need in the art for improved method and apparatus for fixation of implantable devices. Such approaches should provide an explantation option with minimal trauma to the tissue of the patient.

### SUMMARY

The present application relates to method and apparatus for reducing migration and rotation of implantable devices including an expandable element and an elongate portion.

This Summary is an overview of some of the teachings of the present application and is not intended to be an exclusive or exhaustive treatment of the present subject matter. Further details about the present subject matter are found in the detailed description and the appended claims. The scope of the present invention is defined by the appended claims and their legal equivalents.

### BRIEF DESCRIPTION OF THE DRAWINGS

FIG. 1A shows an example of placement of implantable devices with respect to a human bladder and urethra according to one embodiment of the present subject matter.

FIG. 1B demonstrates migration of one of the two implantable devices.

FIG. 1C demonstrates rotation of one expandable element of one of the two implantable devices.

FIG. 2 shows placement of a sleeve adapted to provide ingrowth to provide an adhesion according to one embodiment of the present subject matter.

FIG. 3 shows a closeup of the sleeve of FIG. 2.

FIG. 4 shows a cross section of an implantable device including a sleeve attached using an adhesive according to one embodiment of the present subject matter.

FIG. 5 shows a cross section of surface structure adapted to enhance ingrowth according to one embodiment of the present subject matter.

FIG. 6 shows a cross section of surface structure adapted to enhance ingrowth and resist migration according to one embodiment of the present subject matter.

FIG. 7 shows a cross section of a helical sleeve structure adapted to enhance ingrowth according to one embodiment of the present subject matter.

FIG. 8 shows one example of a tool for removal of a helical sleeve structure according to one embodiment of the present subject matter.

FIG. 9 shows one example of an expandable tool for removal of a helical sleeve structure according to one embodiment of the present subject matter.

FIG. 10 shows one example of a split ring device adapted to enhance ingrowth according to one embodiment of the present subject matter.

FIG. 11 shows one example of a segmented ring device adapted to enhance ingrowth according to one embodiment of the present subject matter.

FIG. 12A shows one example of a hook adapted for fixation according to one embodiment of the present subject matter.

FIG. 12B shows a cross section of a portion of the implantable device of FIG. 12A according to one embodiment of the present subject matter.

FIG. 13 shows one example of a plurality of hooks adapted for fixation according to one embodiment of the present subject matter.

FIGS. 14A and 14B show one example of use of ingrowth-promoting material at a distal end of an apparatus according to one embodiment of the present subject matter.

FIGS. 15A to 15E show some examples of an implantable device featuring a variable stiffness design adapted to reduce rotation according to various embodiments of the present subject matter.

FIG. 16A shows one example of an external stiffener for an implantable device according to one embodiment of the present subject matter.

FIGS. 16B and 16C show one example of an externally coiled stiffener for an implantable device according to one embodiment of the present subject matter.

FIGS. 16D and 16E show different examples of an internally coiled stiffener for an implantable device according to various embodiments of the present subject matter.

FIG. 17 shows one example of a restraint on a proximal bond according to one embodiment of the present subject matter.

FIG. 18A shows one embodiment of an irregular shape of an expandable element of the implantable device according to one embodiment of the present subject matter, as compared to that of FIG. 18B.

FIG. 19 shows one embodiment of a microtextured expandable element according to one embodiment of the present subject matter.

FIG. 20 shows one embodiment of an extra stabilizing balloon for an implantable device according to one embodiment of the present subject matter.

### DETAILED DESCRIPTION

The following detailed description of the present invention refers to subject matter in the accompanying drawings which show, by way of illustration, specific aspects and embodiments in which the present subject matter may be practiced. These embodiments are described in sufficient detail to enable those skilled in the art to practice the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The present subject matter relates to implantable devices which can be placed near a patient's urethra to improve continence. The implantable devices are described in applications such as such as the implantable devices described in U.S. Pat. Nos. 6,045,498, 5,964,806, 6,579,224, and 6,419,624 and their related patents and applications, the descriptions of which are hereby incorporated in their entirety. Such applications also contain information as to the placement of the implantable devices. Placement of such devices is also discussed in U.S. Provisional Patent Application Ser. No. 61/039,738, filed Mar. 26, 2008, entitled: METHOD AND APPARATUS FOR PLACEMENT OF IMPLANTABLE DEVICE ADJACENT A BODY LUMEN, which is incorporated by reference in its entirety.

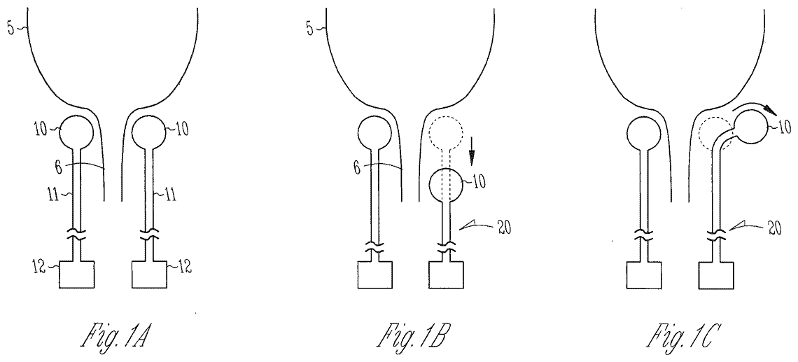

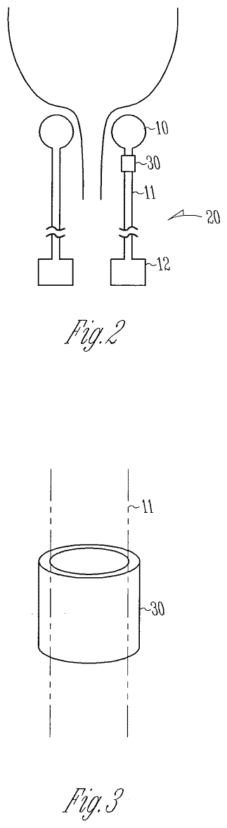

FIG. 1A shows an example of placement of implantable devices with respect to a human bladder and urethra according to one embodiment of the present subject matter. In one embodiment of the implantable devices, an expandable element 10 is connected to an elongate conduit 11 and terminates in a port 12, such as a septum. Typically, the expandable element 10 is implanted proximal the urethra 6 near the bladder 5. Such location is also referred to as the “bladder neck.” The actual positions of the expandable elements 10 of such devices with respect to the urethra 6 and bladder neck may vary; however, FIG. 1A shows the original position of the devices compared to the positions in FIGS. 1B and 1C for purposes of demonstration of the unwanted aspects of migration and rotation.

FIG. 1B demonstrates migration of one of the two implantable devices. Some time after implantation, it is possible to find that the expandable element 10 of implantable device 20 has moved from its original implantation site. In some cases the expandable element 10 will move down the dilation tract formed during implantation of the device. Once it has moved out of position, the implantation may no longer provide adequate tissue bulking in the vicinity of the bladder neck, and may fail to restore continence as needed.

Another unwanted effect that may be possible to find is rotation. FIG. 1C demonstrates rotation of one expandable element of one of the two implantable devices. The expandable element 10 of implantable device 20 is shown as being rotated in position relative to its original placement. The rotation can result in displacement of the expandable element 10, which may reduce the efficacy of the implantation for purposes of enhancing coaptation, and continence. However, rotation can be problematic even in cases where the expandable element 10 is not substantially displaced because the elongate conduit 11 can apply unwanted force on the expandable element 10 and may result in device failure over time due to erosion of material.

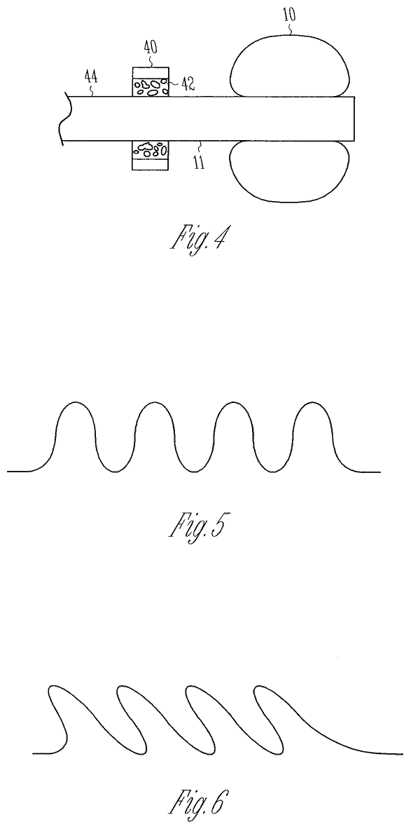

One way to reduce the chances of movement of the expandable element 10 after implantation is to provide a means for adhesion of the implantable device to the tissue in which it is implanted. FIG. 2 shows placement of a sleeve adapted to provide ingrowth to provide an adhesion according to one embodiment of the present subject matter. In one embodiment a small adhesion forms near the end of the tubing closest to the expandable element 10 by the use of an ingrowth collar or sleeve 30. Different materials can be used, including but not limited to EPTFE (expanded polytetrafluoroethelene as used in some vascular grafts) to encircle the elongate conduit 11 near the expandable element 10. The ingrowth collar or sleeve 30 allows the adhesion to form which is a form of fixation of the device to avoid migration and to avoid rotation. Other ingrowth materials may include polypropylene or polyethylene mesh as used for hernia repairs or open cell silicone or polyurethane foams. In various embodiments it is beneficial to use a mesh or foam cell size and/or material and/or surface area selected to make the adhesion of limited strength so that the implantable device 20 can be pulled out without breaking it during removal or causing trauma to surrounding tissue. In one embodiment, the ingrowth sleeve 30 as designed to have less than a 5 lb. pull strength. Other pull strength limits are possible without departing from the scope of the present subject matter. FIG. 3 shows a closeup of the sleeve of FIG. 2, which is friction fit or otherwise secured to the elongate conduit 11 of the implantable device 20 in various embodiments. In certain embodiments the ingrowth material is bioresorbable so that it fixes the device in place after a short time allowing the surrounding tissue to adapt and stabilize the device but would be resorbed over a longer timeframe and allow the device to be easily removed if need be. One example of a bioresorbable material is polylactic acid.

In one embodiment, the implantable device 20 can be removed by cutting off the septum, feeding a cylindrical tool over the implantable device, and using the tool to go over the conduit, cutting the adhesion to facilitate removal of the implantable device at a pull strength that is less than what would destroy the implantable device. The cylindrical tool may also have a slot along its length so that it can be fit over and along the conduit so that the part need not be cut off. In various embodiments, the tool can cut between the elongate conduit and the sleeve or the tool can cut the sleeve away from the tissue. One type of tool to accomplish the cutting is the use of a properly positioned beveled blade. Other tools and approaches are possible without departing from the scope of the present subject matter.

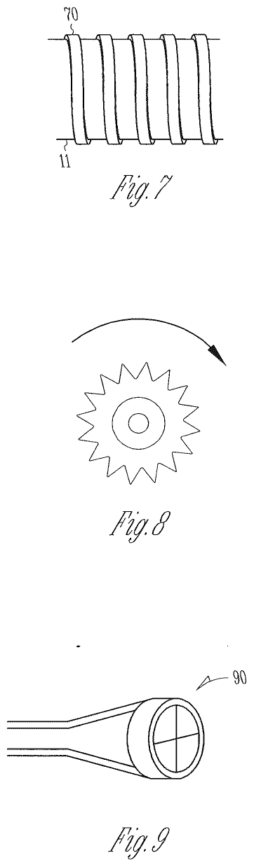

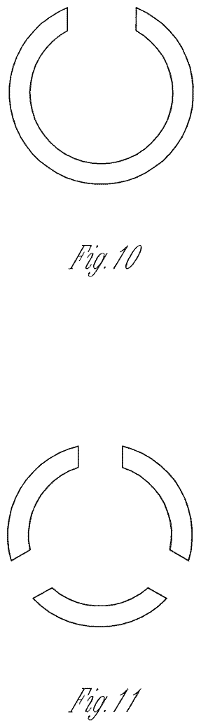

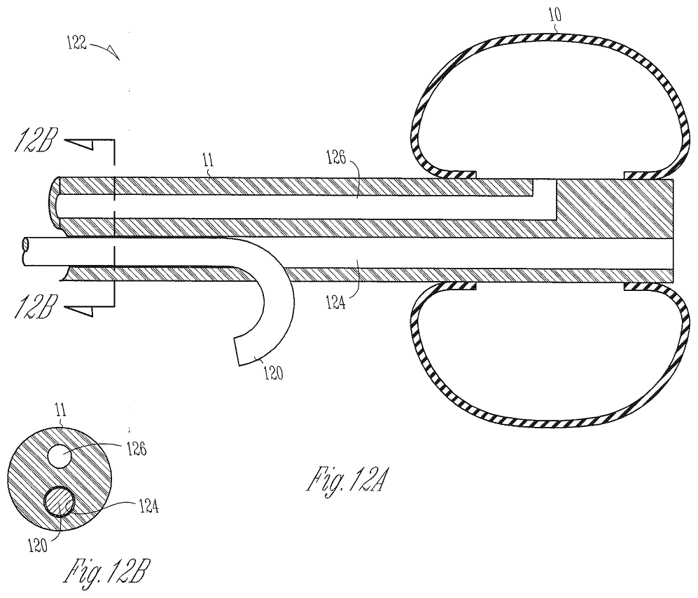

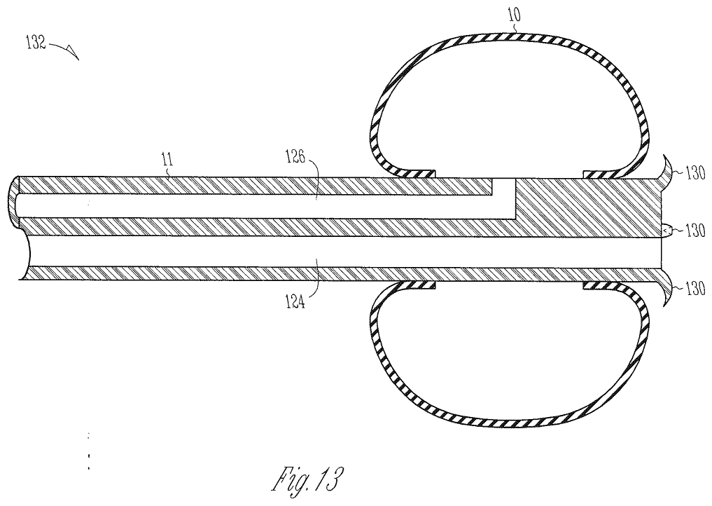

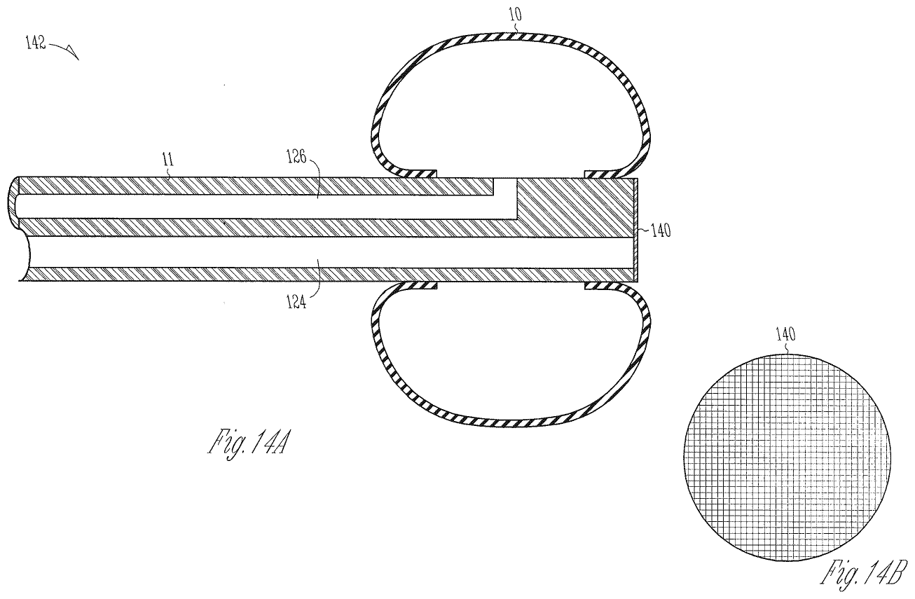

FIG. 4 shows a cross section of an implantable device 44 including a sleeve 40 attached to elongate conduit 11 using an adhesive 42 according to one embodiment of the present subject matter. Such sleeves 40 provide adequate fixation of the implantable device 44 during its implantation. In such embodiments, the adhesive has a tear strength which is less than the pull strength limit of the implantable device 44. Thus, to remove the implantable device 44, the sleeve 40 may stay in place while the glue 42 shears as the implantable device 44 is pulled out of the patient. In such embodiments, the split ring or segmented ring sleeves as shown in FIGS. 10 and 11 may be used to allow the withdrawal of the implantable device. This is especially useful for devices where the cross section at the expandable element 10 is greater than the cross section for the elongate conduit 11. Other types of split ring sleeves may be used without departing from the scope of the present subject matter.

FIG. 5 shows a cross section of surface structure adapted to enhance ingrowth according to one embodiment of the present subject matter. The surface structure can be porous and is selected to provide the proper pull strength, yet retain the implantable device in position during its normal use.

FIG. 6 shows a cross section of surface structure adapted to enhance ingrowth and resist migration according to one embodiment of the present subject matter. The angled nature of the structure forms additional resistance to migrations back down the dialation path.

FIG. 7 shows a cross section of a helical sleeve structure adapted to enhance ingrowth according to one embodiment of the present subject matter. The helical sleeve structure 70 over the elongate conduit 11 acts to fix the implantable device. To remove the device, it is screwed out of the tissue. In one embodiment a pushwire or other rod is used to withdraw the device. The pushwire or rod can be keyed to facilitate application of torque to withdraw the device. In one embodiment, a structure, such as that shown in FIG. 8, is used to lock the pushwire or rod with a channel or other receiver in the implantable device for withdrawal. FIG. 8 shows one example of a tool for removal of a helical sleeve structure according to one embodiment of the present subject matter.

In various embodiments a collet-type device, such as the expanding device shown in FIG. 9 is used to withdraw the device. FIG. 9 shows one example of an expandable tool for removal of a helical sleeve structure according to one embodiment of the present subject matter. A remote actuator (not shown) is used to expand distal ring 90 so as to engage with a lumen or other feature of the implantable device which is to be extracted.

FIG. 12A shows one example of a hook adapted for fixation according to one embodiment of the present subject matter. In one embodiment, after the implantable device 122 is placed in position, a Nitinol hook 120 is threaded through an inner lumen 124 of the elongate conduit 11 of implantable device 122 to serve as a fixation to the local tissue. Other types of hooks may be used without departing from the scope of the present subject matter. The hook is placed near the expandable element 10 of the implantable device 122 to retain it in position. FIG. 12B shows a cross section of elongate conduit showing a cross section of hook 120 in the space of what was the inner lumen 124 and another inner lumen 126 in communication with expandable element 10. In various embodiments inner lumen 126 is connected to a port with a septum (not shown) as described in the various documents incorporated by reference. In various embodiments inner lumen 124 is used with various implantation tools such as a pushrod or other tool to place implantable device 122 in position. Various inner lumens for implantable devices are described in patent documents, including, but not limited to, U.S. Pat. Nos. 6,045,498, 5,964,806, 6,579,224, and 6,419,624 and their related patents and applications, the descriptions of which are hereby incorporated in their entirety. Such applications also contain information as to the placement of the implantable devices. Placement of devices is discussed in various documents, including, but not limited to U.S. Provisional Patent Application Ser. No. 61/039,738, filed Mar. 26, 2008, entitled: METHOD AND APPARATUS FOR PLACEMENT OF IMPLANTABLE DEVICE ADJACENT A BODY LUMEN, which is incorporated by reference in its entirety.

FIG. 13 shows one example of a plurality of hooks adapted for fixation according to one embodiment of the present subject matter. In various embodiments, fixation hooks 130 at the distal tip are deployed to provide fixation to the tissue after the implantable device 132 is placed in its desired position. In various embodiments, the expandable element 10 is expanded and the hooks 130 are recessed to shield bladder from puncturing. Other hook positions, shapes, and numbers are possible without departing from the scope of the present subject matter.

FIGS. 14A and 14B show one example of use of ingrowth-promoting material at a distal end of an implantable device according to one embodiment of the present subject matter. In various embodiments, a patch or coating of ingrowth-promoting material 140 at the distal end of the device 142 provides adhesion at or near the distal end of the device 142. Such approaches can reduce migration and rotation of expandable element 10. In certain embodiments the ingrowth material is bioresorbable so that it fixes the device in place after a short time allowing the surrounding tissue to adapt and stabilize the device but would be resorbed over a longer timeframe and allow the device to be easily removed if need be. One example of a bioresorbable material is polylactic acid. It is understood that various materials, shapes and positions can be used without departing from the scope of the present subject matter.

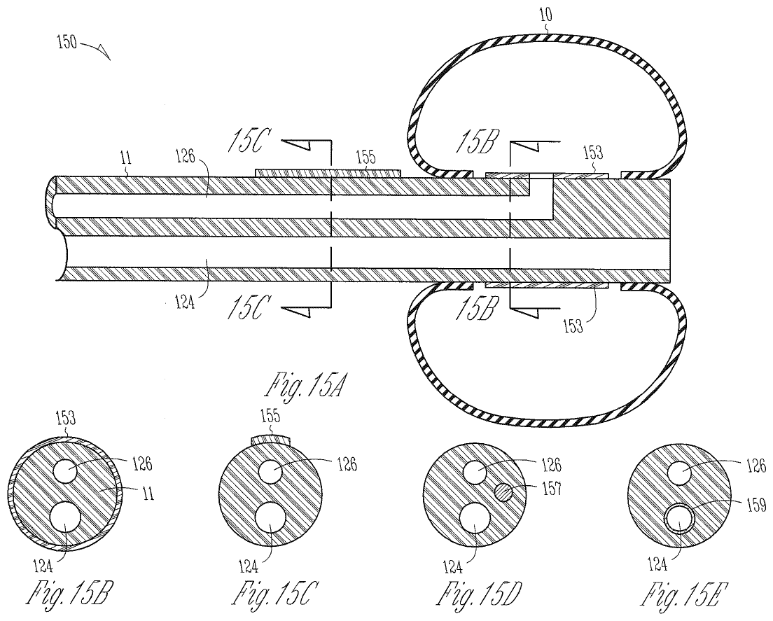

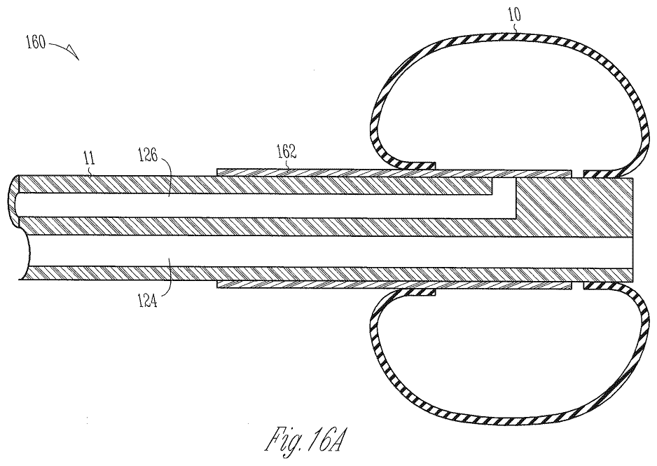

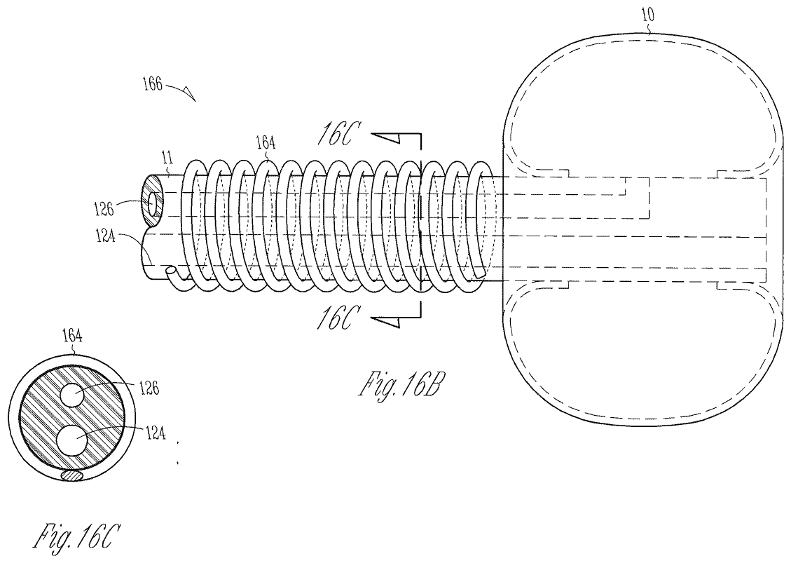

FIGS. 15A-E show some examples of an implantable device featuring a variable stiffness design adapted to reduce rotation according to various embodiments of the present subject matter. In FIG. 15A different stiffeners are shown, which may be used individually, or in combination, in various applications. FIG. 15B is a cross section drawing showing stiffener 153 which is a form of annular ring with an aperture for filling expandable element 10. FIG. 15C is a cross section showing a stiffening member 155. FIG. 15D shows a cross section showing a separate lumen with a stiffening member 157. FIG. 15E shows a cross section with an annular stiffening member 159 placed in inner lumen 124. In some embodiments, expansion of the expandable element 10 can also make tubing in central portion stretch and can also have a preferential force on the tubing and may cause it to rotate. In various embodiments a stiffener of varying stiffness can be inserted in a lumen of the elongate conduit to provide more stiffening at the distal end than at the proximal end of the implantable device 150. In one application, a stiffener 157 is added to a third central lumen of the implantable device after it is deployed into position. In varying embodiments, the stiffening effect is built into the implantable device as it is manufactured. In various embodiments, a stiffener 159 is placed in the same lumen 124 that is used to position the implantable device. This could be done by the surgeon after removing the pushwire at time of implant. Other types of stiffeners and deployments are possible without departing from the scope of the present subject matter.

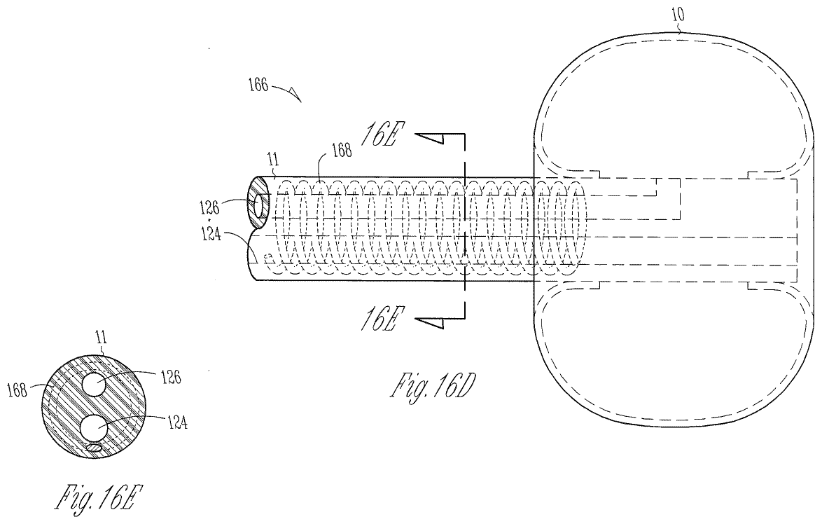

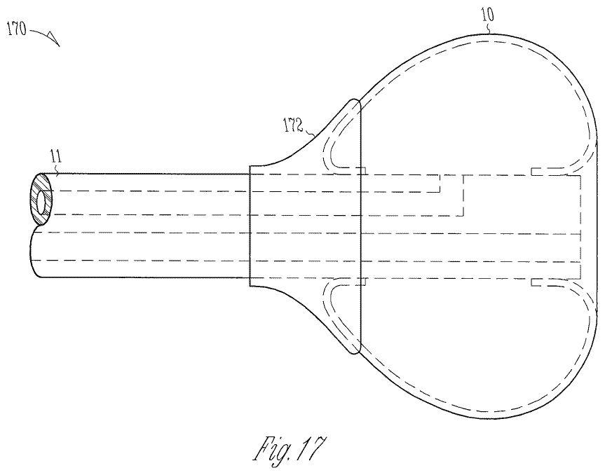

FIG. 16A shows one example of an external stiffener for an implantable device according to one embodiment of the present subject matter. In such embodiments, sleeve 162 provides additional stiffness at the proximal bond area of elongate conduit 11 of the expandable element 10. In various embodiments, the sleeve 162 continues into the expandable element 10 portion of the elongate conduit 11. In various embodiments the sleeve 162 is manufactured as an integral portion of elongate conduit 11.

In various embodiments, a lubricious coating, sleeve, or material can be positioned anywhere along the elongate conduit or about the external stiffener in embodiments which employ a stiffener, or both, to reduce or eliminate wear should the expandable element 10 come in contact with the stiffener and/or the elongate conduit. In such events, the lubricious coating, sleeve, or material would prevent wear of the expandable element 10.

FIGS. 16B and 16C show one example of an externally coiled stiffener for an implantable device according to one embodiment of the present subject matter. As shown in FIGS. 16B and 16C, coil 164 can be placed outside of elongate conduit 11. A coil can also be displaced within the elongate conduit 11 as shown as coil 168 in FIGS. 16D and 16E. Coils 164 and 168 provide additional stiffness to prevent rotation of the expandable element 10 of implantable device 166.

FIG. 17 shows one example of a restraint on a proximal bond according to one embodiment of the present subject matter. The restraint 172 on the proximal bond of the expandable element 10 to elongate conduit 11 reduces or eliminates wear and/or erosion on the proximal bond. In various embodiments, the restraint 172 is a collar that is attached to the implantable device 170. In various embodiments, the restraint 172 is generated by one or more additional dips in the coating process that produces the implantable device 170. In various embodiments, the restraint provides asymmetric balloon inflation of the expandable element 10. Other shapes and constraints and asymmetries are possible without departing from the scope of the present subject matter.

Expandable elements with asymmetrical shapes can be engineered to reduce the possibility of contact with the elongate conduit by the expandable element. For example, the bulge shown in the example of FIG. 17 is larger at the distal end of the expandable element, which can reduce the tendency of the expandable element to come in contact with the elongate conduit.

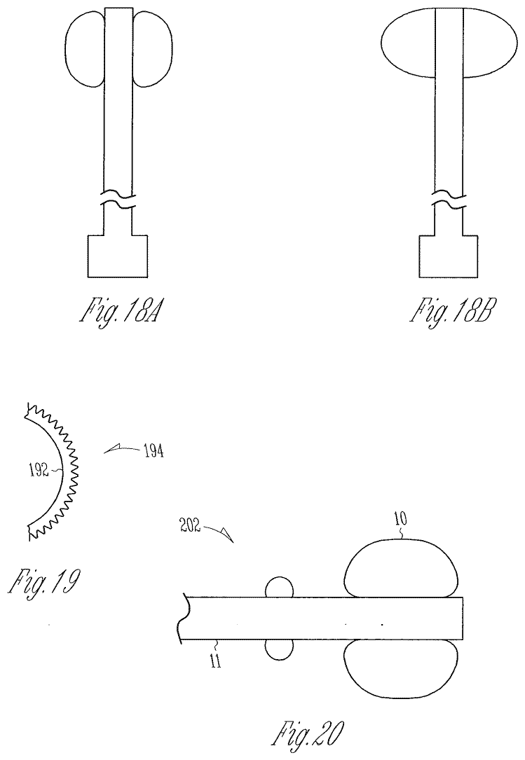

FIG. 18A shows one embodiment of an irregular shape of an expandable element of the implantable device according to one embodiment of the present subject matter, as compared to that of FIG. 18B. The irregular shape is made squarer than that of FIG. 18B to avoid rotation. Various different shapes may be used without departing from the scope of the present subject matter.

FIG. 19 shows one embodiment of a microtextured expandable element according to one embodiment of the present subject matter. A portion of the wall of expandable element 192 is microtextured 194 to reduce rotation. A tool can be employed to remove the expandable element 10.

FIG. 20 shows one embodiment of an extra stabilizing balloon for an implantable device according to one embodiment of the present subject matter. Balloon 202 is placed to prevent the implantable device from migrating, and may avoid rotation by holding the elongate conduit in place and thereby limit the expandable element 10 from rotating.

It is understood that various combinations of the foregoing aspects of the present subject matter can be combined to provide apparatus having multiple benefits.

This application is intended to cover adaptations and variations of the present subject matter. It is to be understood that the above description is intended to be illustrative, and not restrictive. The scope of the present subject matter should be determined with reference to the appended claim, along with the full scope of legal equivalents to which the claims are entitled.

1. (canceled)

2. An implantable device for implantation into tissue adjacent to a urethra connected to a bladder, comprising:
an expandable balloon capable of expansion in the tissue adjacent to the urethra;
an elongate conduit having a distal portion connected to the expandable balloon and a proximal portion, the elongate conduit including a first lumen in fluid communication with the expandable balloon to allow for filling of the expandable balloon; and
a stiffener connected to the distal portion of the elongate conduit to reduce rotation of the implantable device after the implantation.

3. The implantable device of claim 2, wherein the stiffener is connected to the distal portion of the elongate conduit and inside the expandable balloon, the stiffener comprising an annular member including an aperture positioned to allow the expandable balloon to be filled through the first lumen.

4. The implantable device of claim 2, wherein the elongate conduit comprises a second lumen configured to accommodate an implantation tool configured to place the implantable device in position during implantation of the implantable device.

5. The implantable device of claim 4, wherein the elongate conduit comprises a third lumen, and the stiffener comprises a stiffening member placed in the third lumen.

6. The implantable device of claim 5, wherein the stiffening member has varying stiffness and is configured to provide more stiffening at a distal end of the implantable device than at a proximal end of the implantable device.

7. The implantable device of claim 5, wherein the stiffener comprises an annular stiffening member placed in the second lumen.

8. The implantable device of claim 4, wherein the stiffener is configured to be placed in the second lumen after the implantable device is placed in position.

9. The implantable device of claim 2, wherein the stiffener is connected to the distal portion of the elongate conduit and positioned outside the expandable balloon.

10. The implantable device of claim 2, wherein the stiffener comprises a coil on the elongate conduit.

11. The implantable device of claim 2, wherein the stiffener comprises a coil disposed inside of the elongate conduit.

12. The implantable device of claim 2, wherein the expandable balloon comprises a wall including a portion microtextured to further reduce the rotation of the implantable device after the implantation.

13. The implantable device of claim 2, further comprising an ingrowth collar or sleeve encircling the elongate conduit near the expandable balloon and made of a bioresorbable material, the ingrowth collar or sleeve configured to allow ingrowth to provide adhesion of the implantable device to the tissue adjacent to the urethra.

14. The implantable device of claim 2, further comprising a helical sleeve structure on the elongate conduit.

15. The implantable device of claim 2, further comprising one or more hooks configured to fix the implantable device to the tissue adjacent the urethra.

16. The implantable device of claim 15, wherein the elongate conduit comprises a second lumen, and the one or more hooks comprises a Nitinol hook configured to be deployed by being threaded through the second lumen.

17. The implantable device of claim 15, wherein the one or more hooks comprise a plurality of hooks at a distal tip of the elongate conduit.

18. A method of treating incontinence in a human having a bladder, a urethra connected to the bladder, and tissue adjacent the urethra, the method comprising:
improving continence of the human expanding a balloon of an implantable device implanted in the human with the balloon placed in the tissue adjacent the urethra, the implantable device including the balloon and an elongate conduit having a distal portion connected to the balloon and a proximal portion, the elongate conduit including a lumen in fluid communication with the balloon to allow for filling of the balloon; and
reducing rotation of the implantable device after being placed in the human by using a stiffener of the implantable device.

19. The method of claim 18, wherein using the stiffener comprises using an annular stiffening member connected to the distal portion of the elongate conduit, disposed inside the balloon, and including an aperture positioned to allow the balloon to be filled through the lumen.

20. The method of claim 18, wherein the elongate conduit comprises an additional lumen configured to accommodate a tool configured to place the implantable device in position during implantation of the implantable device, and using the stiffener comprises placing the stiffener after the implantable device is placed in position.

21. The method of claim 18, further comprising reducing migration of the implantable device in the human by using one or more hooks of the implantable device.
